(19) United States
(12) Patent Application Publication
(10) Pub. No.: US 2006/0287593 A1
(43) Pub. Date: Dec. 21, 2006

(54) System and method providing communication in a medical imaging system

(75) Inventors: Srinivas Kiran Jaggu, Bangalore (IN); Girish Laxmankumar Sannellappanavar, Dharwar (IN); Rajeev R. Marar, Waukesha, WI (US); Dalesh S. Dharamshi, Bangalore (IN); Lakshmi N. Gudapakkam, Algonquin, IL (US)

Correspondence Address: PATRICK W. RASCHE (12553 - 1000), ARMSTRONG TEASDALE LLP, ONE METROPOLITAN SQUARE, SUITE 2600, ST. LOUIS, MO, 63102-2740, US

(73) Assignee: General Electric Company

(21) Appl. No.: 11/157,099

(22) Filed: Jun. 20, 2005

(51) Int. Cl. A61B 5/05

(52) U.S. Cl. 600/407

(57) ABSTRACT

Systems and methods providing a communication system for a medical imaging system are provided. The communication system includes a plurality of user interfaces within the medical imaging system for receiving user inputs. The communication system further includes at least one router for controlling communications of the plurality of user interfaces to control at least one subsystem of the medical imaging system based on the received user inputs.

### BACKGROUND OF THE INVENTION

The invention relates generally to medical imaging systems, and more particularly, to a communication system for a medical imaging system.

Medical imaging systems such as Computer Tomography (CT) scanners, X-ray scanners, Magnetic Resonance Imaging (MRI) scanners, Positron Emission Tomography (PET) scanner, Ultrasound scanners, and the like, are defined by a number of subsystems that communicate to provide the major functionalities for these systems. Examples of such subsystems of an X-ray scanner include, for example, an X-ray generator, a table positioner, a system control and an operator console. These subsystems are typically different physical computer systems running on different operating systems.

Some functionalities of a medical imaging system may be critical and need deterministic responses in order to function properly. For example, during image acquisition, the image detection and controlling must have quick and deterministic responses. If not, problems such as overdose may occur. However, other functionalities, such as operator console operations need to be more user friendly. The former requirements are typically supported by a real-time operating system and the latter requirements are typically supported by a general purpose operating system. Therefore, in a medical imaging system, typically one or more subsystems may run on a real-time operating system and others may run on a general purpose operating system. Operating systems include, for example, Windows NT™, Windows 2000™ of Microsoft Corporation, Red Hat Linux™ of Red Hat, which may form a general purpose operating system, and VxWorkS™ of WindRiver for the real-time operations.

The subsystems may be further subdivided into software applications or other logical subsystems. These software applications are typically object oriented programs that individually or in combination with other software applications perform a specific functionality. For example, the functionality can be image acquisition, image processing, etc.

The medical imaging system further may include a plurality of user interfaces or devices, through which a user, for example, a medical technician or technologist, communicates and controls the medical imaging system. For example, the communication with an X-ray scanner may involve positioning of the scanner table, setting the field of view for the exposure, stopping the acquisition, etc. Further, user interfaces or devices may be present at different physical locations and/or operated by different users trying to control the same functionality. For example, in an X-ray scanner, a user interface may be located inside the room where the X-ray is exposed and another user interface may be located in the control room where the technologists and technicians may view live scanning at a safe distance to prevent X-ray exposure. However, known medical imaging systems do not allow user interfaces or devices to simultaneously control the medical imaging system.

Furthermore, a user interface or a device typically has to interact with other software and/or hardware components to perform specific functionality. Therefore, in known medical imaging systems, if the architecture is modified by adding a new device or functionality, or by modifying the existing ones, then all the other software and/or hardware components may have to be modified. Hence, the complexity of maintenance and likelihood of errors or operating problems increase.

### BRIEF DESCRIPTION OF THE INVENTION

In one exemplary embodiment, a communication system for a medical imaging system is provided. The communication system includes a plurality of user interfaces within the medical imaging system for receiving user inputs. The communication system further includes at least one router for controlling communications of the plurality of user interfaces to control at least one subsystem of the medical imaging system based on the received user inputs.

In another exemplary embodiment, a method for controlling communication in a medical imaging system is provided. The method includes receiving a plurality of requests from a plurality of user interfaces of a medical imaging system. The method further includes controlling routing of the plurality of requests within the medical imaging system to control the medical imaging system.

### BRIEF DESCRIPTION OF THE DRAWINGS

FIG. 1 is a block diagram of a communication system for a medical imaging system in accordance with an exemplary embodiment of the invention.

FIG. 2 is a block diagram of a physical subsystem in accordance with an exemplary embodiment of the invention that may be used in connection with the medical imaging system shown in FIG. 1.

FIG. 3 is a block diagram of a physical subsystem in accordance with another exemplary embodiment of the invention that may be used in connection with the medical imaging system shown in FIG. 1.

FIG. 4 is a flowchart illustrating a method for controlling communication in a medical imaging system in accordance with an exemplary embodiment of the invention.

FIGS. 5A, 5B and 5C is a flowchart illustrating a method for controlling communication in a medical imaging system in accordance with another exemplary embodiment of the invention.

### DETAILED DESCRIPTION OF THE INVENTION

Various embodiments of the invention provide a communication system in a medical imaging system. The medical imaging system may be, for example, a Computer Tomography (CT) scanner, a Medical Resonance Imaging (MRI) Scanner, a Positron Emission Tomography (PET) scanner, an ultrasound scanner, and/or an X-ray scanner.

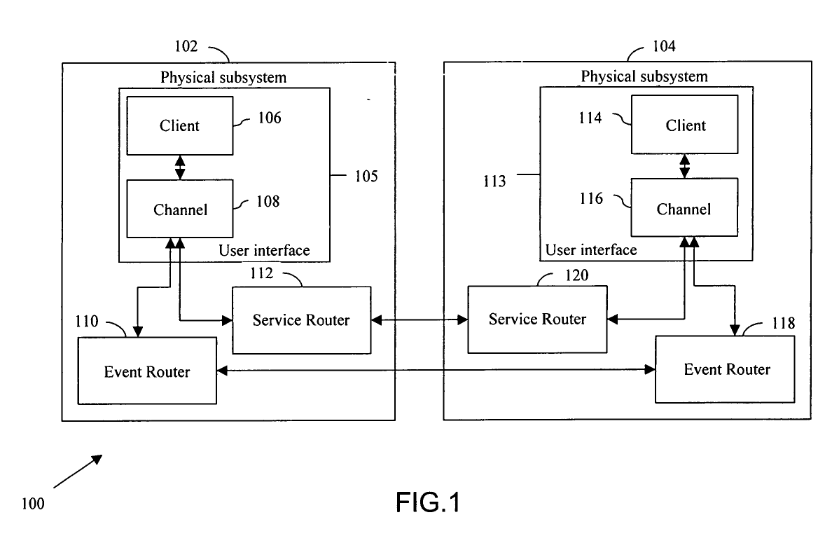

FIG. 1 shows a communication system in a medical imaging system 100 in accordance with various embodiments of the invention. Medical imaging system 100 may include a plurality of physical subsystems. In an exemplary embodiment, medical imaging system 100 includes a physical subsystem 102 and a physical subsystem 104. However, additional or fewer physical subsystems may be provided. It should be noted that these physical subsystems relate to, but are not limited to, components and/or entities that perform different operations or functions in medical imaging system 100. The physical subsystems may be, for example, physical computer systems running on different operating systems. For example, in an X-ray scanner, a physical subsystem may be an X-ray generator, a table positioner, a system control or an operator console.

In various embodiments of the invention, physical subsystem 102 includes a user interface 105. User interface 105 includes one or more client components (referred to herein as clients) clients 106 and one or more channels 108. In addition, physical subsystem 102 further includes an event router 110 and a service router 112.

In operation, a user of medical imaging system 100 may request user interface 105 for access to an application. In various embodiments of the invention, this request may be, for example, for changing a parameter of medical imaging system 100. For example, in an X-ray scanner, the user may want or need to change various values for scanning. (e.g., kV and mA values). User interface 105 sends the request to client 106. Client 106, in various embodiments of the invention corresponds to user interface 105 (e.g., software component associated with user interface 105). Client 106 then sends the request to channel 108. Channel 108, based on the request, directs the request to one of an event router 110 and a service router 112. Depending on the request, event router 110 and/or service router 112 then controls the routing of the request.

The user interface according to various embodiments of the invention relates to, but is not limited to, an interface through which the user may communicate with the software components of medical imaging system 100. The software components may further interact with the hardware components to perform specific functionalities. For example, in setting the kV and mA values of an X-ray scanner using the user interface, the user interface has to communicate with the generator to provide the desired exposure. In various embodiments of the invention, user interface 105 may be, for example, a software based graphical user interface on a visual display unit controlled using a mouse and a keyboard, a touch screen user interface, etc. The physical subsystems further may include a plurality of user interfaces similar to user interface 105.

Client 106 registers (e.g., logs in) on event router 110 and service router 112, and receives an identification, which is used for future communication with event router 110 and service router 112. Further, client 106 provides an interface for event router 110 and service router 112 to communicate with user interface 105. Client 106 also communicates with other similar clients in a distributed environment through event router 110 and service router 112. Client 106 also provides access to the available set of applications of user interface 105 and allows other clients in medical imaging system 100 access to the set of applications. In various embodiments, other clients do not access the applications by directly communicating with client 106. In these various embodiments, other clients send a request to event router 110 and service router 112. Event router 110 and service router 112 then communicate with client 106, through channel 108. Client 106 may be associated with any other device or services in medical imaging system 100. Further, in various embodiments, user interface 105 may be associated with a plurality of clients.

In various embodiments of the invention, the interface provided by client 106 is defined using, for example, Interface Definition Language (IDL) available from Object Management Group, Inc. (OMG), of Framingham, Mass. With IDL, objects may be implemented/constructed in various programming languages and may communicate with each other. An object generally refers to a software component that may be used to build arrangements of variables and operations therein. These objects may be used, for example, to build client 106. Independence of IDL is achieved by, for example, mapping the IDL to multiple programming languages such as C, C++, and Java.

In operation, the requests sent by user interface 105 to client 106 may be asynchronous requests or synchronous requests. The asynchronous request may be, for example, a request for changing the operation mode of an X-ray scanner (e.g., changing the X-ray scanning mode from the chest scanning mode to the shoulder scanning mode). The synchronous request may be, for example, a request for printing. In various embodiments of the invention, client 106 directs the requests (asynchronous or synchronous) to channel 108. In one exemplary embodiment, channel 108 directs asynchronous requests of client 106 to event router 110, and synchronous requests to service router 112. Further, client 106 also communicates with channel 108 to enable the communication between pluralities of components of user interface 105. For example, if in response to activating (e.g., pressing) a radio button on user interface 105, another button should also be activated then such a request is communicated by client 106 to channel 108.

In various embodiments of the invention, event router 110 handles asynchronous requests (asynchronous operations) from channel 108. Client 106 through channel 108 communicates asynchronous requests to other clients through event router 110. In various embodiments of the invention, client 106 may publish (e.g., communicate to a plurality of clients 106) notifications (through event router 110) for the user and/or other clients, in response to an asynchronous request. Further, client 106 subscribes for notifications of interest to event router 110. In response to a publication (of a notification) from client 106, event router 110 publishes the notification on all clients (of medical imaging system 100) that subscribed for the notification. In various embodiments of the invention, the format of the notification is pre-determined and is provided in OMG's IDL. For example, in response to a request for changing the operation mode of an X-ray scanner, all the related clients (of medical imaging system 100) update their operation modes and publish notifications regarding the update. Thereafter, event router 110 publishes the notification regarding the update on all the related clients (that have subscribed for the notification).

In various embodiments of the invention, service router 112 handles synchronous requests (synchronous operations) from channel 108. The synchronous operation enables client 106 to request another client to perform, for example, a particular function or operation. In various embodiments of the invention, client 106 does not identify the actual client that performs the application, thereby providing transparency in medical imaging system 100. Client 106 provides a list of functions and/or operations relating to, for example, user interfaces, devices or services available from client and provides these to service router 112. For example, if client 106 relates to print server, then client 106 may provide a print service. When a requesting client requests a function or operation, service router 112 directs the request to the client corresponding to, for example, the application that can provide the requested function or operation based on the stored information for each client. The request is then processed by the client corresponding to the application. In this example, if a request client requests a print service, service router 112 then directs the request to client 106. Client 106 in response processes the request. In various embodiments of the invention, service router 112 may find a client corresponding to the application to process the requests of client 106, as well as other clients in physical subsystem 102. In various embodiments of the invention, if an application provider is not available, then service router 112 provides a call back to the requesting client on the availability of the application provider.

Similar to physical subsystem 102, physical subsystem 104 includes a user interface 113, an event router 118 and a service router 120. User interface 113 includes client 114 and channel 116. The components of physical subsystem 104, namely, user interface 113, event router 118 and service router 120, and have similar properties and functioning as the components of physical subsystem 102.

Service router 112 and service router 120 communicate with each other to control the routing of requests in medical imaging system 100. Similarly, event router 110 and event router 118 communicate with each other. For example, client 114 transmits a request to service router 120 through channel 116 and the requested action may be performed by client 106 in physical subsystem 102. To process the request, service router 120 communicates with service router 112. Service router 112 then requests client 106 to perform the requested operation or function. Service router 112 communicates with client 106 through channel 108 to perform the requested operation or function. Client 106 then performs the requested operation or function and may send the result to service router 112 through channel 108. Service router 112 communicates the result to service router 120. Service router 120 then communicates the result to client 114 through channel 116.

In various embodiments of the present invention, the link (in medical imaging system 100) between physical subsystem 102 and components of physical subsystem 102, physical subsystem 104 and components of physical subsystem 104 and physical subsystem 102 and physical subsystem 104 is enabled through a communication link that provides platform (operating system) independent interoperability between a plurality of physical subsystems. In various embodiments of the invention, the communication link is, for example, enabled through a Shared Memory Communication, or a Common Object Request Broker Architecture (CORBA®) based architecture.

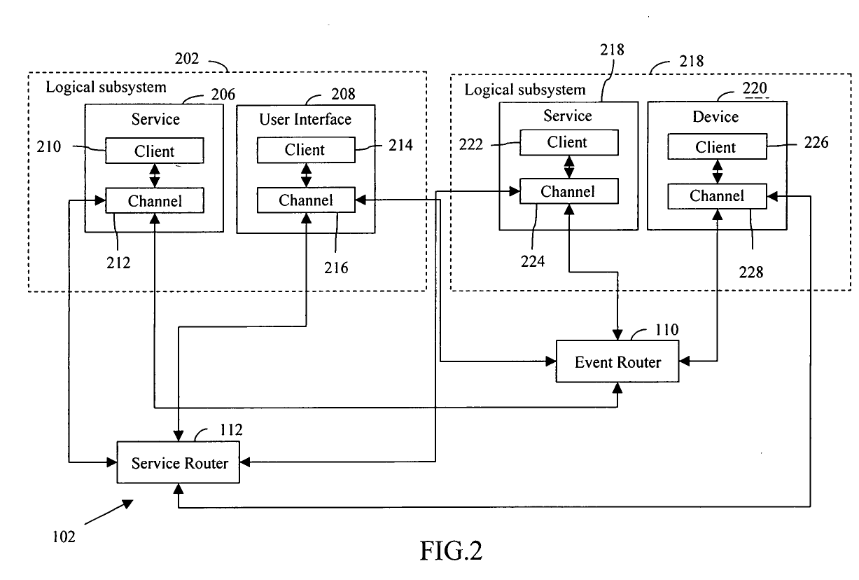

In addition to applications for performing a requested operation or function, physical subsystem 102 further may include a device or a service for performing the requested operation or function. The device may be, but is not limited to, a machine, device and/or interface through which a user may communicate with the software components of medical imaging system 100, and the software components may further communicate with hardware components to perform specific functionality. Examples of the device include a hand switch, special keys on a keyboard of a user interface, etc. The service may be, but is not limited to, an object-oriented software entity or a hardware component performing a specific application in physical subsystem 102. For example, in an X-ray scanner, there may be an application director or controller that manages the physical subsystem 102 and physical subsystem 104 during acquisition of an image. Further, in various embodiments of the invention, the clients also may be associated with other applications, devices or services. This is further illustrated in FIG. 2.

FIG. 2 is a block diagram of physical subsystem 102 in accordance with an exemplary embodiment of the invention that may be used in connection with medical imaging system 100 in shown FIG. 1. Physical subsystem 102 further may include a plurality of logical subsystems. For example, physical subsystem 102 may include a logical subsystem 202 and a logical subsystem 204. A logical subsystem generally refers to, but is not limited to, a module containing one or more user interfaces, devices or services, that may be dependent on each other in order to perform a specific action in medical imaging system 100. The logical subsystem, for example, may be an operator console application of X-ray scanner. Examples of applications are an image processing subsystem, a positioner subsystem, and a system control subsystem. It should be noted that the logical subsystems may be configured to encompass more than one physical subsystem.

In various embodiments of the invention, logical subsystem 202 includes a service 206 and a user interface 208. Service 206 includes a corresponding client 210 and a channel 212 and user interface 208 includes a corresponding client 214 and a channel 216. Further, logical subsystem 204 includes a service 218 and a device 220. Service 218 includes a client 222 and a channel 224 and device 220 includes a client 226 and a channel 228.

The user interfaces, the services or the devices may be dependent on each other. For example, service 206 and user interface 208 in logical subsystem 202 may be dependent on each other. The user may request a change to a parameter of physical subsystem 102 of medical imaging system 100 through user interface 208. Client 214 (of user interface 208) sends the request to service router 112 through channel 216. Service router 112 then requests service 206 to change the parameter, which then changes the parameter. Thereafter, service router 112 communicates, for example, a success notification to client 214. Therefore, even though service 206 and user interface 208 are not directly communicating with each other, they are dependent on each other. Similarly, service 218 and device 220 may be dependent on each other.

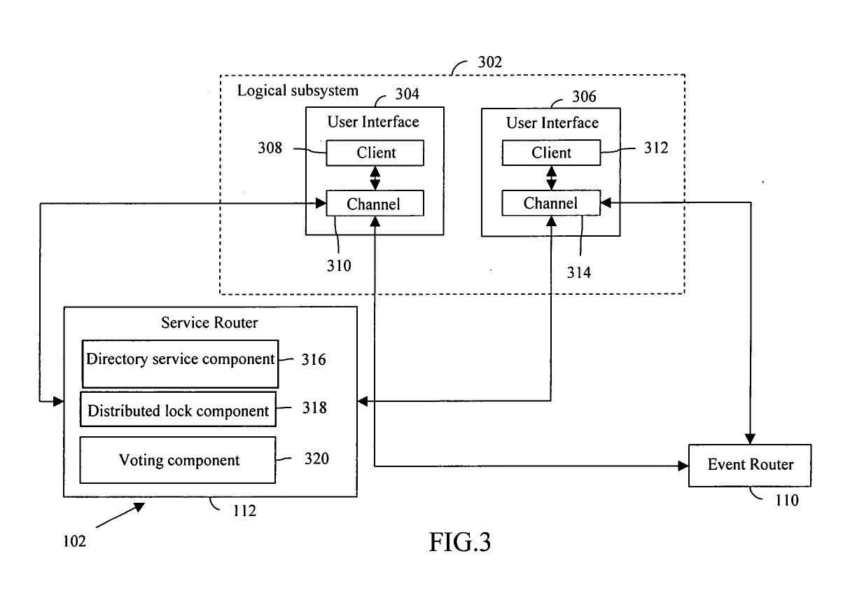

It should be noted that a logical subsystem may include a plurality of services, user interfaces and devices that are dependent on each other. This inter-relationship and management are further illustrated in FIG. 3.

FIG. 3 is a block diagram of physical subsystem 102 in accordance with another exemplary embodiment of the invention that may be used in connection with medical imaging system 100 in shown in FIG. 1. Physical subsystem 102 includes a logical subsystem 302. Logical subsystem 302 further includes a user interface 304 and a user interface 306. User interface 304 includes a client 308 and a channel 310 and user interface 306 includes a client 312 and a channel 314. Service router 112 further includes a directory service component 316, a voting component 318 and a distributed lock component 320.

In various embodiments of the invention, in response to a request from client 308 (through channel 310), directory service component 316 locates and identifies a client, for example, corresponding to an application provider (e.g., application that can provide the requested operation or function) to process the request. In various embodiments of the invention, service router 112 may locate a client corresponding to the application to process the requests of client 106, as well as other clients in physical subsystem 102.

User interface 304 and user interface 306 may provide identical operations and functions and may perform the same applications from different physical locations. For example, in an X-ray scanner, a user interface can be located inside the room where the X-ray is exposed and another user interface can be located in the control room where the technologists and technicians may match the live scanning at a safe distance to prevent X-ray exposure. It may be possible that user interface 304 and user interface 306 may, simultaneously, (through client 308 and client 312, respectively) send requests to change the same parameter of physical subsystem 102 from different physical locations. Thus, the parameters for the requested change may be made from multiple user interfaces or devices.

In such cases, the first client (the client sending the first request to change the parameter to service router 112) requests a vote from voting component 318. Voting component 318 then makes synchronous calls on all the clients that are interested in or may be affected by the change of the value of the parameter. If all the clients agree to the change, then voting component 318 processes the request of the first client. For example, if client 308 is the first client to send a request to change the value of a parameter, then client 308 requests for a vote through voting component 318. If client 312 agrees to the change, then only the request from client 308 is processed, otherwise a failure notification is communicated to client 308.

User interface 304 and user interface 306 may simultaneously (through client 308 and client 312, respectively) send requests to change parameters that are different, but are in a same group (e.g., correspond to related components). A group may be defined as, but is not limited to, a set of parameters that are related. For example, in an X-ray scanner, kV and mA values are related. A change in kV value causes a corresponding change in mA value. One interface may also request a change of a parameter while the parameter is being changed based on a request from another interface. Further, in another example, image properties of medical imaging system 100 such as, for example, image contrast and gray scale levels are related.

In such a case, first client (the client sending the first request to change the parameter to service router 112) receives priority from distributed lock component 320 of service router 112 and locks the system with respect to the parameter to be changed (e.g., no further change allowed). Distributed lock component 320 does not allow any other clients to change the parameter or a related parameter until the request of first client is processed or released. For example, if client 308 is the first client to send the request, then client 308 receives priority through distributed lock component 320. Client 312 (which may also be requesting a change of the same or the related parameter) can change the value of the parameter only after the request of client 308 is processed.

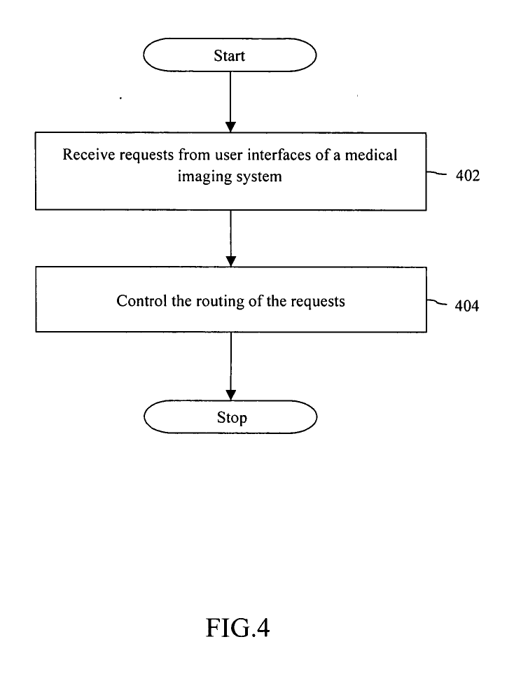

FIG. 4 is a flowchart illustrating a method for controlling communication in medical imaging system 100 (shown in FIG. 1) in accordance with an exemplary embodiment of the invention. The method is described with respect to physical subsystem 102 (shown in FIG. 1) of medical imaging system 100. However, the method may be implemented in connection with any other physical subsystem in medical imaging system 100. The user, via user interface 105 (shown in FIG. 1), requests a function, operation or use of an application, etc. The request may be an asynchronous request or synchronous request. At 402, one of event router 110 (shown in FIG. 1) and service router 112 (shown in FIG. 1) receives the request from user interface 105 of medical imaging system 100. User interface 105 communicates the request through client 106 (shown in FIG. 1), which transmits the request to channel 108 (shown in FIG. 1). Channel 108, then communicates the request to one of event router 110 and service router 112. If the request is asynchronous, channel 108 sends the request to event router 110; otherwise channel 108 sends the request to service router 112. At 404, one of event router 110 and service router 112 controls the routing of the request. The event router 110 or service router 112 communicates with other routers of medical imaging system 100, if required, and obtains the request processed by the relevant client. Thereafter, user interface 105 receives a success notification.

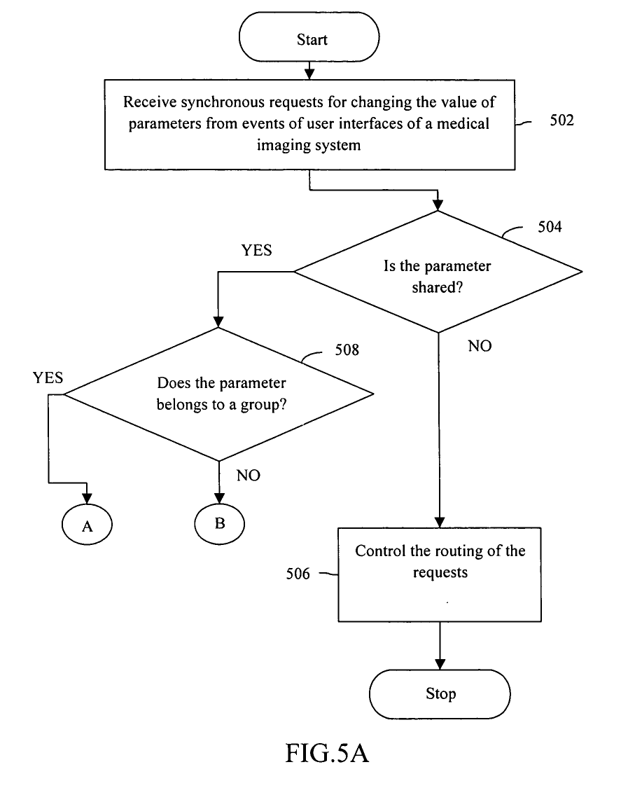

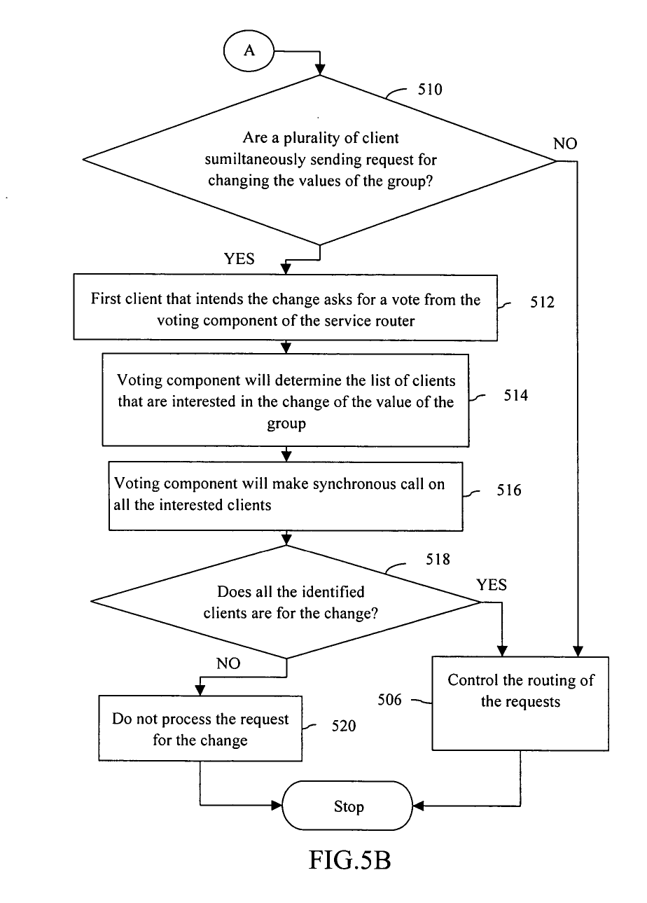

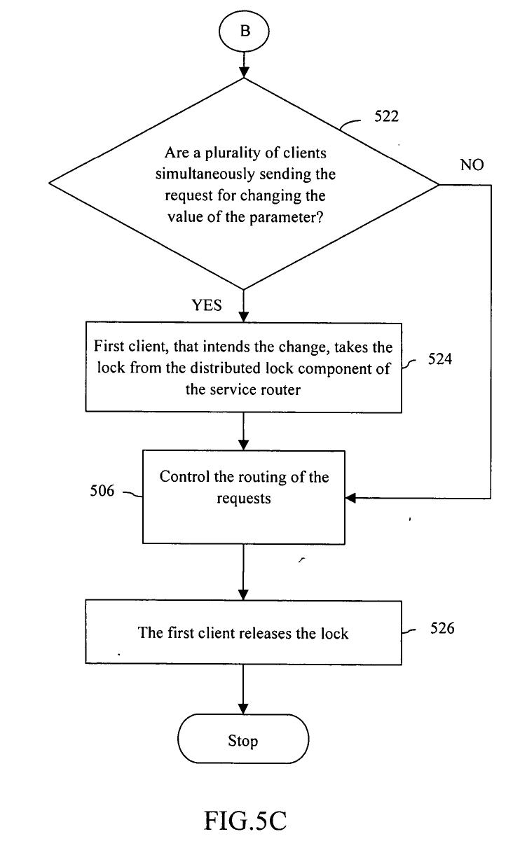

FIGS. 5A, 5B and 5C is a flowchart illustrating a method for controlling communication in medical imaging system 100 (shown in FIG. 1) in accordance with another embodiment of the invention. The method is described with respect to physical subsystem 102 (shown in FIG. 1) of medical imaging system 100 but may be implemented in connection with any subsystem in medical imaging system 100. A user requests, via user interface 304, (shown in FIG. 3) a change in the value of a parameter related to physical subsystem 102. The request may be a synchronous request. At 502, service router 112 (shown in FIG. 1) receives the request (e.g., a synchronous request) from user interface 304. User interface 304 communicates the synchronous request through client 308 (shown in FIG. 3) and client 308 communicates to the synchronous request to service router 112 through channel 310 (shown in FIG. 3). At 504, a determination is made as to whether the parameter is shared, (e.g. a determination is made as to whether the synchronous request for change in value of the parameter can be made from multiple user interfaces or devices). If the parameter is not shared, service router 112 controls the routing of the request at 506. For example, service router 112 communicates with other routers of medical imaging system 100, if required, and the synchronous request is processed by the relevant client. Thereafter, user interface 304 receives a success notification.

However, if the parameter is shared, a determination is made at 508 as to whether the parameter belongs to a group, i.e., a check is made as to whether the parameter belongs to a group of related parameters. If the parameter does not belong to a group, a determination is made at 510 as to whether pluralities of clients are simultaneously sending the request (e.g., synchronous request) for changing the value of the parameter. If, for example, only user interface 304 (shown in FIG. 3) has sent the request for changing the value of the parameter, service router 112 (shown in FIG. 1) controls the routing of the request at 506. Service router 112 communicates with other routers of medical imaging system 100 (shown in FIG. 1), if required, and the request is processed by the relevant client. Thereafter, user interface 304 receives a success notification.

If, at 510, a plurality of clients are simultaneously sending the requests (e.g., synchronous requests) to change the value of the parameter, the first client (the client that sending the first request to change the parameter to service router 112 (shown in FIG. 1)) requests a vote for the change from voting component 318 of service router 112 at 512. For example, client 312 (of user interface 306 (shown in FIG. 3)) may send a request for a change in the value of a parameter simultaneously with client 308 (of user interface 304 (shown in FIG. 3)). Assuming client 308 is the first client to send the request, client 308 then requests a vote from voting component 318. At 514, voting component 318 of service router 112 determines the list of clients that are interested in or affected by the change of the value of the parameter. In the present example, client 312 of user interface 306 is identified as interested in the change in the value of the parameter. At 516, voting component 318 of service router 112 makes synchronous calls on all the interested clients. Voting component 318 queries all the interested clients whether they agree or disagree with the change proposed by the first client. In this example, client 312 receives a synchronous call from voting component 318 of service router 112. At 518, a determination is made as to whether all interested clients agree to the change. If all the interested clients do not agree to the change, the synchronous request for the change is not processed at 520. In the present example, if client 312 does not agree to the change, the request of client 308 is not processed. Thereafter, user interface 304 (through client 308) receives a failure notification.

If all the interested clients agree to the change, service router 112 (shown in FIG. 1) controls the routing of the request at 506. Service router 112 communicates with other routers of medical imaging system 100 (shown in FIG. 1), if required, and the request is processed by the relevant client. Thereafter, user interface 304 (through client 308 (shown in FIG. 3)) receives a success notification.

However, if at 508 it is determined that the parameter belongs to a group, at 522 a determination is made as to whether a plurality of clients are simultaneously sending the synchronous request for changing the value of the group. If, for example, only user interface 304 (shown in FIG. 3) makes the request to change the values of the group, service router 112 controls the routing of the request. Service router 112 communicates with other routers of medical imaging system 100, if required, and the request is processed by the relevant client. Thereafter, user interface 304 receives a success notification.

If, at 522, a plurality of clients are simultaneously sending the request for changing the values of the group, first client (sending the first request to change the values of the group to service router 112 (shown in FIG. 1)) processes the values of the group corresponding to the requested change in the value of the parameter. At 524, the first client then sends the processed values of the group to service router 112 and receives a priority lock from distributed lock component 320 of service router 112. For example, client 312 (of user interface 306 (shown in FIG. 3)) may send a request for the change in the values of the group (by sending the request for the change in the same parameter or a related parameter) simultaneously with client 308 (of user interface 304 (shown in FIG. 3)). Client 308 is the first client to send the request. Client 308 then processes the values of the group and then communicates the values of the group to service router 112. Client 308 then receives a priority lock such that the values of the group are locked from change by requests from other clients. Service router 112 in response controls the routing of the request at 506. Service router 112 communicates with other routers of medical imaging system 100 (shown in FIG. 1), if required, and the request is processed by the relevant client. Thereafter, user interface 304 (of client 308) receives a success notification. The first client then releases the priority lock at 526. In this example, client 308 releases the priority lock.

In various embodiments of the invention, clients may dynamically add or remove functions, operations, associated applications etc. from medical imaging system 100. When a client dynamically adds, for example, an application to medical imaging system 100, the client only has to re-register itself to the corresponding physical subsystem event router and service router.

The various embodiments of the invention provide a communication system for a medical imaging system that may be used across all platforms and languages. The medical imaging system has a common framework that may be used on different operating systems both real-time and general purpose. Improved maintainability of the medical imaging system is thereby provided. Further, various embodiments of the invention provide a communication system for a medical imaging system that is scalable. The communication system allows changing architectures having different physical subsystem without redesigning the framework.

The various embodiments of the invention also provide a medical imaging system that allows multiple user interfaces and devices to communicate and control the medical imaging system at the same time or at different times. Thus, simultaneous control of the medical imaging system may be provided.

A technical effect of various embodiments of the invention is to provide a scaleable communication system for a medical imaging system. The communication system allows changing architectures having different physical subsystem without redesigning the framework. Another technical effect of various embodiments of the invention to allow multiple user interfaces and devices to control (e.g., simultaneous control) the medical imaging system. Yet another technical effect of various embodiments of the invention to allow clients to dynamically add or remove, for example, an associated application.

The various embodiments or components thereof may be implemented as part of a computer system. The computer system may include a computer, an input device, a display unit and an interface, for example, for accessing the Internet. The computer may include a microprocessor. The microprocessor may be connected to a communication bus. The computer may also include a memory. The memory may include Random Access Memory (RAM) and Read Only Memory (ROM). The computer system further may include a storage device, which may be a hard disk drive or a removable storage drive such as a floppy disk drive, optical disk drive, and the like. The storage device can also be other similar means for loading computer programs or other instructions into the computer system.

The computer system executes a set of instructions that are stored in one or more storage elements, in order to process input data. The storage elements may also hold data or other information as desired or needed. The storage element may be in the form of an information source or a physical memory element within the processing machine.

The set of instructions may include various commands that instruct the processing machine to perform specific operations such as the processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

While the invention has been described in terms of various specific embodiments, those skilled in the art will recognize that the invention can be practiced with modification within the spirit and scope of the claims.

What is claimed is:

1. A communication system for a medical imaging system, said communication system comprising:
a plurality of user interfaces within a medical imaging system for receiving user inputs; and
at least one router for controlling communications of the plurality of user interfaces to control at least one subsystem of the medical imaging system based on the received user inputs.

2. A communication system in accordance with claim 1 wherein the at least one subsystem comprises at least one physical subsystem.

3. A communication system in accordance with claim 1 wherein the at least one subsystem comprises at least one logical subsystem.

4. A communication system in accordance with claim 1 wherein the at least one router is configured to control synchronous operations.

5. A communication system in accordance with claim 1 wherein the at least one router is configured to control asynchronous operations.

6. A communication system in accordance with claim 1 further comprising a plurality of client components corresponding to the plurality of user interfaces.

7. A communication system in accordance with claim 1 wherein at least one subsystem comprises at least one device and further comprising a client component corresponding to the at least one device.

8. A communication system in accordance with claim 1 further comprising a plurality of client components corresponding to a plurality of services associated with the medical imaging system.

9. A communication system in accordance with claim 1 further comprising a channel corresponding to each of a component of the subsystem.

10. A communication system in accordance with claim 1 further comprising a directory service component for identifying services within the medical imaging system.

11. A communication system in accordance with claim 1 further comprising a distributed lock component configured to block a communication from at least one user interface, wherein the plurality of user interfaces are simultaneously communicating for controlling the medical imaging system.

12. A communication system in accordance with claim 1 further comprising a voting component configured to receive user inputs corresponding to a request to change an operating parameter of the medical imaging system, wherein the plurality of user interfaces are simultaneously communicating for controlling the medical imaging system.

13. A method for controlling communication in a medical imaging system, said method comprising:
receiving a plurality of requests from a plurality of user interfaces of a medical imaging system; and
controlling routing of the plurality of requests within the medical imaging system to control the medical imaging system.

14. A method in accordance with claim 13 further comprising associating a client component with each of the plurality of user interfaces.

15. A method in accordance with claim 13 further comprising associating a client component with at least one of a device and a service of the medical imaging system.

16. A method in accordance with claim 15 further comprising at least one of dynamically adding and removing at least one of an associated device and an associated service.

17. A method in accordance with claim 13 wherein a client component is associated with at least one of (i) each of the plurality of user interfaces (ii) devices of the medical imaging system and (iii) services of the medical imaging system, and further comprising registering each client component for identification.

18. A method in accordance with claim 13 wherein the controlling comprises determining at least one of available devices and services of the medical imaging system corresponding to at least one of the plurality of requests.

19. A method in accordance with claim 13 further comprising receiving user responses to requests for a change in an operating parameter of the medical imaging system to control the medical imaging system.

20. A method in accordance with claim 19 further comprising changing the operating parameter upon receiving acceptance responses from the plurality of user interfaces, a number of acceptance responses to be received before changing the operating parameter being determined dynamically.

21. A method in accordance with claim 20 further comprising notifying the user interface requesting the change upon receiving the acceptance responses.

22. A method in accordance with claim 13 wherein controlling routing of the plurality of requests comprises blocking communication of a request to change an operating parameter of the medical imaging system based on a previous requested change of the operating parameter.

23. A method in accordance with claim 22 wherein the requested change is communicated while the previous requested change is being processed.

24. A method in accordance with claim 22 further comprising allowing communication of the requested change based on a user input received from a user interface communicating the previous requested change.

25. A computer-readable media having computer-executable components comprising:
a plurality of client components associated with a plurality of user interfaces of a medical imaging system; and
a router component for controlling communications of the plurality of client components to control at least one subsystem of the medical imaging system based on user inputs received by the user interfaces.
